(19) United States
(12) Patent Application Publication
(10) Pub. No.: US 2007/0292889 A1
(43) Pub. Date: Dec. 20, 2007

(54) Immunoassay magnetic trapping device

(75) Inventors: Christopher G. Bailey, Pleasanton, CA (US); Perry M. Bell, Tracy, CA (US); Kodumudi S. Venkateswaran, Livermore, CA (US); Allen T. Christian, Madison, WI (US); Elizabeth K. Wheeler, Livermore, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/455,211

(22) Filed: Jun. 16, 2006

(51) Int. Cl. G01N 33/53; C12M 3/00; G01N 33/551

(52) U.S. Cl. 435/7.1; 436/524; 435/287.2

(57) ABSTRACT

A system for immunoassaying a sample comprising providing magnetic beads, connecting signal molecules to the beads, connecting the sample to the magnetic beads with the connected signal molecules, magnetically trapping the magnetic beads with the connected signal molecules and the sample, lysising the sample, and analyzing the sample.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to biological assays and more particularly to an immunoassay magnetic trapping device.

2. State of Technology

U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005 provides the following state of technology information, “The most commonly employed portable pathogen detection is strip-type tests, such as those used in handheld glucose diagnostics or the Joint Biological Point Detection System (JBPDS), a system used for detection of biowarfare agents. These tests are held or ‘smart ticket’ assay, and are currently the smallest embodiment of a viable pathogen detection technology. In the JBPDS, for example, a membrane strip is printed with three lines: a mobile line of colored latex particles coated with an antibody to the bioagent being detected, a fixed line of a second antibody to the same bioagent, and a fixed line of antibody directed to the antibody on blue latex particles. To perform an assay, a liquid sample is added to the device that hydrates the latex spheres (which are located in the sample well). If the targeted bioagent is present, a complex is formed between the latex sphere and bioagent. This complex wicks through the strip and is captured by the fixed line of antibody to the bioagent forming a visible line of color. A line will also appear at the next fixed line due to capture of free latex spheres. Thus a negative assay will only have a single line at the control line and a positive assay will have two lines. The JBPDS obtains multiplex capability by delivering multiple ‘tickets’ (printed membrane strips) to the assay by means of a mechanical carousel. Currently, nine different ‘tickets,’ each sensitive to a different bioagent, share the sample and perform the analysis with fluidic automation and photonic inspection of the test lines. This technology represents a credible solution for military use since the number of target pathogens is limited. For civilian use, however, the scaling of the device to 30 or more pathogens is quite problematic. The carousel becomes increasingly complicated and large, while dividing the sample between the different assays creates an unacceptable reduction in sensitivity.”

In an article titled, “U.S. Is Deploying a Monitor System for Germ Attacks,” by Judith Miller in The New York Times on Jan. 22, 2003, it was reported, “To help protect against the threat of bioterrorism, the Bush administration on Wednesday will start deploying a national system of environmental monitors that is intended to tell within 24 hours whether anthrax, smallpox and other deadly germs have been released into the air, senior administration officials said today. The system uses advanced data analysis that officials said had been quietly adapted since the September 11 attacks and tested over the past nine months. It will adapt many of the Environmental Protection Agency's 3,000 air quality monitoring stations throughout the country to register unusual quantities of a wide range of pathogens that cause diseases that incapacitate and kill . . . . The new environmental surveillance system uses monitoring technology and methods developed in part by the Department of Energy's national laboratories. Samples of DNA are analyzed using polymerase chain reaction techniques, which examine the genetic signatures of the organisms in a sample, and make rapid and accurate evaluations of that organism . . . . Officials who helped develop the system said that tests performed at Dugway Proving Ground in Utah and national laboratories showed that the system would almost certainly detect the deliberate release of several of the most dangerous pathogens. ‘Obviously, the larger the release, the greater the probability that the agent will be detected,’ an official said. ‘But given the coverage provided by the E.P.A. system, even a small release, depending on which way the wind was blowing and other meteorological conditions, is likely to be picked up.’”

In an article titled, “Biodetectors Evolving, Monitoring U.S. Cities,” by Sally Cole in the May 2003 issue of Homeland Security Solutions, it was reported, “The anthrax letter attacks of 2001, and subsequent deaths of five people, brought home the reality of bioterrorism to Americans and provided a wake-up call for the U.S. government about the need for a method to detect and mitigate the impact of any such future attacks. Long before the anthrax letter attacks, scientists at two of the U.S. Department of Energy's national laboratories, Lawrence Livermore National Laboratory (LLNL) and Los Alamos National Laboratory (LANL), were busy pioneering a ‘biodetector’ akin to a smoke detector to rapidly detect the criminal use of biological agents. This technology is now expected to play a large role in the U.S. government's recently unveiled homeland security counter-terrorism initiative, Bio-Watch, which is designed to detect airborne bioterrorist attacks on major U.S. cities within hours. Announced back in January, Bio-Watch is a multi-faceted, multi-agency program that involves the U.S. Department of Energy, the Environmental Protection Agency (EPA), and the U.S. Department of Health and Human Services' Centers for Disease Control and Prevention (CDC). Many of the EPA's 3,000 air-quality monitoring stations throughout the country are being adapted with biodetectors to register unusual quantities of a wide range of pathogens that cause diseases that incapacitate and kill, according to the EPA. The nationwide network of environmental monitors and biodetectors, which reportedly will eventually monitor more than 120 U.S. cities, is expected to detect and report a biological attack within 24 hours. Citing security reasons, the EPA declined to disclose further details about the program at this time . . . . The Autonomous Pathogen Detection System (APDS) is a file-cabinet-sized machine that sucks in air, runs tests, and reports the results itself. APDS integrates a flow cytometer and real-time PCR detector with sample collection, sample preparation, and fluidics to provide a compact, autonomously operating instrument capable of simultaneously detecting multiple pathogens and/or toxins. ‘The system is designed for fixed locations,’ says Langlois, ‘where it continuously monitors air samples and automatically reports the presence of specific biological agents. APDS is targeted for domestic applications in which the public is at high risk of exposure to covert releases of bioagents—subway systems, transportation terminals, large office complexes, and convention centers . . . . APDS provides the ability to measure up to 100 different agents and controls in a single sample,’ Langlois says. ‘It's being used in public buildings right now.’ The latest evolution of the biodetector, APDS-II, uses bead-capture immunoassays and a compact flow cytometer for the simultaneous identification of multiple biological simulants. Laboratory tests have demonstrated the fully autonomous operation of APDS-II for as long as 24 hours.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for immunoassaying a sample. The system comprises providing magnetic beads, connecting signal molecules to the beads, connecting the sample to the magnetic beads with the connected signal molecules, magnetically trapping the magnetic beads with the connected signal molecules and the sample, lysising the sample, and analyzing the sample. In one embodiment, the present invention provides an immunoassay apparatus for assaying a sample comprising a channel, magnetic beads, signal molecules connected to the beads, a magnetic bead based reagent delivery unit connected to the channel that delivers the magnetic beads and the signal molecules to the channel, a magnet operatively connected to the channel, a lysis unit connected to the channel, a sample delivery unit connected to the channel, at least one reagent delivery unit connected to the channel, at least one wash delivery unit connected to the channel, and an analysis unit connected to the channel.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates one embodiment of an immunoassay device constructed in accordance with the present invention.

FIG. 2 illustrates another embodiment of an immunoassay device constructed in accordance with the present invention.

FIG. 3 illustrates yet another embodiment of an immunoassay device constructed in accordance with the present invention.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Many biochemical assays are performed using reagents immobilized on beads. Additional liquid reactants and wash fluids are added to the bead based sample and are removed manually, which can be slow and imprecise. Alternatively, fluids are left behind causing dilution, which decreases the sensitivity of an assay. Beads are often removed from a sample according to size using vacuum filtration. However, this step leads to sample loss on the filter membrane or reduction in sensitivity of the assay because of excessive backwash volumes needed to remove beads. The use of a flow through magnetic trap allows beads to be removed from a large quantity of sample and concentrated into a much smaller volume, increasing assay sensitivity. While the beads are trapped, chemical reactions and washing steps can be carried out that generate a signal indicative of the quantity of a specific analyte. The use of the indirect signal mechanism enables assays for multiple components to be performed simultaneously. It also allows detection to occur in preferred reagents rather than in the original sample, which may contain interferents.

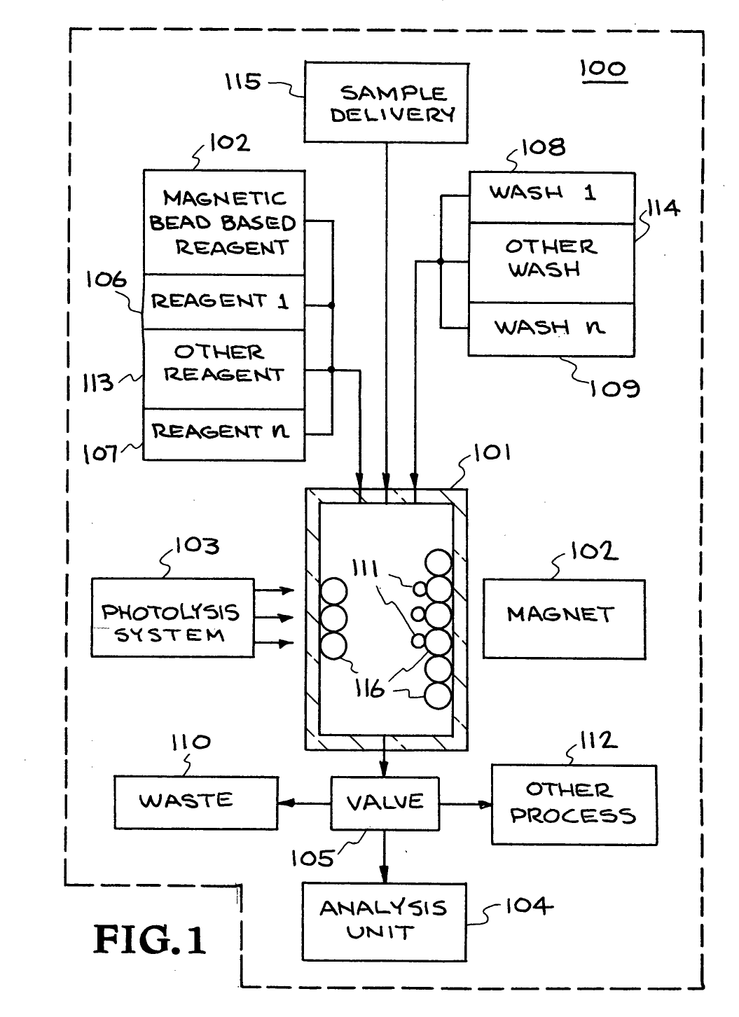

Referring now to FIG. 1, one embodiment of an immunoassay device constructed in accordance with the present invention is illustrated. The immunoassay device is indicated generally by reference numeral 100. The immunoassay device 100 is a device that provides biochemical assays. The immunoassay device 100 can, for example, be the type of immunoassay device described and illustrated in U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005. U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005 is incorporated herein by this reference.

The immunoassay device 100 utilizes a channel 101 through which fluids can be transported. A magnet 102 is positioned adjacent the channel 101. The channel 101 and magnet 102 provide an immunoassay magnetic trapping device. A photolysis unit 103 is positioned adjacent the channel 101 proximate the magnet 102.

A magnetic bead based reagent delivery unit 102 directs a magnetic bead based reagent into the channel 101. A sample is also directed into the channel 101 by the sample delivery unit 115. An individual reagent, or a reagent mix, is also directed into the channel 101. The reagent, or reagent mix, is produced by reagent delivery unit 106 for delivering Reagent 1 and/or reagent delivery unit 113 for delivering other reagents and/or reagent delivery unit 107 for delivering reagent n. The units 106, 113, and 107 allow an individual reagent or a reagent mix comprising reagents 1 through reagent n to be delivered into channel 101.

Signal molecules 111 are connected to the beads 116. The signal molecules can, for example, be eTags available from Monogram Biosciences, Inc., 345 Oyster Point Blvd., South San Francisco, Calif. 94080-1913. The signal molecules 111 can be other signal molecules custom made or commercially available. The signal molecules 111 are released from trapped reagents using a cleaving process based on one or more physical or chemical processes.

A valve 105 downstream of the trapping region directs the flow of reagents to a waste stream 110, to an analysis unit 104, or to some other process area 112. The analysis unit 104 is a device that provides a bio-analysis. Detection of the signal molecules 111 is performed using any type of physical or chemical process, including but not limited to fluorescence, absorption, light scattering, electrochemical processes, conductivity, or mass spectrometry. The analysis unit 104 can, for example, be the type of device described and illustrated in U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005. U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005 is incorporated herein by this reference.

A wash, or a wash mix, is also directed into the channel 101. The wash, or wash mix, is produced by wash unit 108 (Wash 1) and/or other wash unit 114 and/or wash unit 109 (Wash n). The units 108, 114, and 109 allow an individual wash or a wash mix comprising wash 1 through wash n to be delivered into channel 101.

The immunoassay device 100 utilizes the channel 101 through which the fluids are transported. The magnet 102 is positioned adjacent the channel 101. The channel 101 and magnet 102 provide an immunoassay magnetic trapping device. The immunoassay device 100 allows biological assays to be performed using a bead based format. In the past, these were most frequently done in a static, batch configuration and exchange of reagents and washing steps performed manually. Each of the steps can dilute a sample so that the limit of detection for an assay is adversely affected. In the immunoassay device 100 flow through the magnetic trap allows rapid, efficient capture of magnetic bead based reagents, and can be used for pre concentration and sample clean up. Reagents and wash fluids flow past the captured sample and are sent to waste so that no dilution occurs in the assay. After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be removed from the trapping region by withdrawing the permanent magnet or shutting off the electromagnet. Spent magnetic beads can then be flushed from the trapping region using a pressure driven or electrophoretic flow. Removal of the beads prepares the system for another analysis with little cross contamination between samples.

The structural details of the immunoassay magnetic trapping device 100 having been described, the operation of the immunoassay magnetic trapping device 100 will now be considered. Flow through the immunoassay magnetic trapping device 100 allows rapid, efficient capture of magnetic bead based reagents 102, and can be used for pre concentration and sample clean up. Reagents 106 through 107 and wash fluids 108 through 109 can flow past the captured sample 111 and be sent to waste 102 so that no dilution occurs in the assay. It is to be understood that between 106 and 107 or 108 and 109 any number of additional fluid steps can be included.

After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be “removed” or “withdrawn” as needed. Spent magnetic beads can then be flushed from the trapping region using a pressure driven or electrophoretic flow. Removal of the beads prepares the system for another analysis with little cross contamination between samples.

The general processes of the immunoassay magnetic trapping device 100 are the following:
1). Reagents immobilized on magnetic beads flow into the magnetic trap region. In the case of an immunoassay, the immobilized reagent is an antibody. With the magnetic field turned on in the trapping region, beads are removed from solution and captured.
2). A sample stream flows past the captured, immobilized reagents. Molecules with an affinity for the immobilized reagents, antigens in the immunoassay case, will be captured. Those that do not have such affinity will flow to waste. A large volume of sample can be processed in this way with the molecules of interest being captured and concentrated in a small volume.
3). Additional reactive streams are introduced into the trapping region. In the case of an eTag based immunoassay, this could be an eTag bound to an antibody. Alternatively, the immobilized reagents can be washed with water or other fluids to improve the stringency of the assay.
4). Any number of reactive streams or wash steps similar to 3) can be carried out.
5). Signal molecules are removed from the trapped reagents by a cleaving step and sent to the analysis region. For example, eTags can be freed by exposing the immobilized reagent complex to 680 nm light and sent to a capillary electrophoresis, laser induced fluorescence detection system.
6). The magnetic field is removed from the trapping region and all reagents are flushed to waste. The magnetic field is removed by translating the permanent magnet away from the flow channel or turning off the current in the electromagnet. Channels can be rinsed with water, bleach, detergent, or other cleaning fluids to minimize sample cross contamination. The system can then perform another analysis.

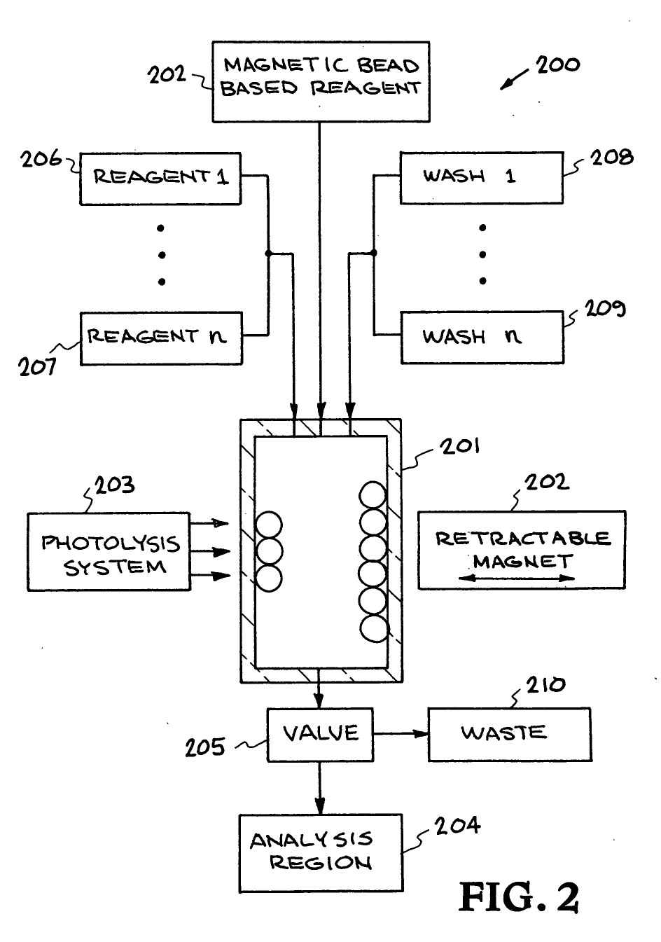

Referring now to FIG. 2, another embodiment of an immunoassay device constructed in accordance with the present invention is illustrated. The immunoassay device is indicated generally by reference numeral 200. The immunoassay device 200 is a device that provides biochemical assays. The immunoassay device 200 can, for example, be the type of immunoassay device described and illustrated in U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005. U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005 is incorporated herein by this reference.

The immunoassay device 200 utilizes a channel 201 through which fluids can be transported. A magnet 202 is positioned adjacent the channel 201. The magnet 202 can be a permanent magnet that can be moved into or withdrawn from the region of the channel. The permanent magnet may be composed of magnetizable iron, NdFeB, SmCo, or other material. The magnet 202 can be positioned near or away from the channel using mechanical actuation. The channel 201 and magnet 202 provide an immunoassay magnetic trapping device. A photolysis unit 203 is positioned adjacent the channel 201 proximate the magnet 202.

A magnetic bead based reagent delivery unit 202 directs a magnetic bead based reagent into the channel 201. A sample is also directed into the channel 201 by the sample delivery unit 215. An individual reagent, or a reagent mix, is also directed into the channel 201. The reagent, or reagent mix, is produced by reagent delivery unit 206 for delivering Reagent 1 and/or reagent delivery unit 207 for delivering reagent n. The units 206 and 207 allow an individual reagent or a reagent mix comprising reagents 1 through reagent n to be delivered into channel 201. It is to be understood that additional reagent delivery units for delivering additional reagents can be added.

Signal molecules are connected to the beads providing a bead signal molecule combination 211. The signal molecules can, for example, comprise eTags available from Monogram Biosciences, Inc., 345 Oyster Point Blvd., South San Francisco, Calif. 94080-1913. The signal molecules 211 can be other signal molecules custom made or commercially available. The signal molecules 211 are released from trapped reagents using a cleaving process based on a one or more physical or chemical processes.

A valve 205 downstream of the trapping region directs the flow of reagents to a waste stream 210, to an analysis unit 204, or to some other process area. The analysis unit 204 is a device that provides a bio-analysis. Detection of the signal molecules 211 is performed using any type of physical or chemical process, including but not limited to fluorescence, absorption, light scattering, electrochemical processes, conductivity, or mass spectrometry. The analysis unit 204 can, for example, be the type of device described and illustrated in U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005. U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005 is incorporated herein by this reference.

A wash, or a wash mix, is also directed into the channel 201. The wash, or wash mix, is produced by wash unit 208 (Wash 1) and/or other wash unit and/or wash unit 209 (Wash n). The units 208 and 209 allow an individual wash or a wash mix comprising wash 1 through wash n to be delivered into channel 201.

The immunoassay device 200 utilizes the channel 201 through which the fluids are transported. The magnet 202 is positioned adjacent the channel 201. The channel 101 and magnet 202 provide an immunoassay magnetic trapping device. The immunoassay device 200 allows biological assays to be performed using a bead based format. In the past, these were most frequently done in a static, batch configuration and exchange of reagents and washing steps performed manually. Each of the steps can dilute a sample so that the limit of detection for an assay is adversely affected. In the immunoassay device 200 flow through the magnetic trap allows rapid, efficient capture of magnetic bead based reagents, and can be used for pre concentration and sample clean up. Reagents and wash fluids flow past the captured sample and are sent to waste so that no dilution occurs in the assay. After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be removed from the trapping region by withdrawing the permanent magnet or shutting off the electromagnet. Spent magnetic beads can then be flushed from the trapping region using a pressure driven or electrophoretic flow. Removal of the beads prepares the system for another analysis with little cross contamination between samples.

The structural details of the immunoassay magnetic trapping device 100 having been described, the operation of the immunoassay magnetic trapping device 200 will now be considered. Flow through the immunoassay magnetic trapping device 200 allows rapid, efficient capture of magnetic bead based reagents 202, and can be used for pre concentration and sample clean up. Reagents 206 through 207 and wash fluids 208 through 209 can flow past the captured sample, beads/signal molecules 211 and be sent to waste 202 so that no dilution occurs in the assay. It is to be understood that between 206 and 207 or 208 and 209 any number of additional fluid steps can be included.

After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be “removed” or “withdrawn” as needed. Spent magnetic beads can then be flushed from the trapping region using a pressure driven or electrophoretic flow. Removal of the beads prepares the system for another analysis with little cross contamination between samples.

The general processes of the immunoassay magnetic trapping device 200 are the following:
1). Reagents immobilized on magnetic beads flow into the magnetic trap region. In the case of an immunoassay, the immobilized reagent is an antibody. With the magnetic field turned on in the trapping region, beads are removed from solution and captured.
2). A sample stream flows past the captured, immobilized reagents. Molecules with an affinity for the immobilized reagents, antigens in the immunoassay case, will be captured. Those that do not have such affinity will flow to waste. A large volume of sample can be processed in this way with the molecules of interest being captured and concentrated in a small volume.
3). Additional reactive streams are introduced into the trapping region. In the case of an eTag based immunoassay, this could be an eTag bound to an antibody. Alternatively, the immobilized reagents can be washed with water or other fluids to improve the stringency of the assay.
4). Any number of reactive streams or wash steps similar to 3) can be carried out.
5). Signal molecules are removed from the trapped reagents by a cleaving step and sent to the analysis region. For example, eTags can be freed by exposing the immobilized reagent complex to 680 nm light and sent to a capillary electrophoresis, laser induced fluorescence detection system.
6). The magnetic field is removed from the trapping region and all reagents are flushed to waste. The magnetic field is removed by translating the permanent magnet away from the flow channel or turning off the current in the electromagnet. Channels can be rinsed with water, bleach, detergent, or other cleaning fluids to minimize sample cross contamination. The system can then perform another analysis.

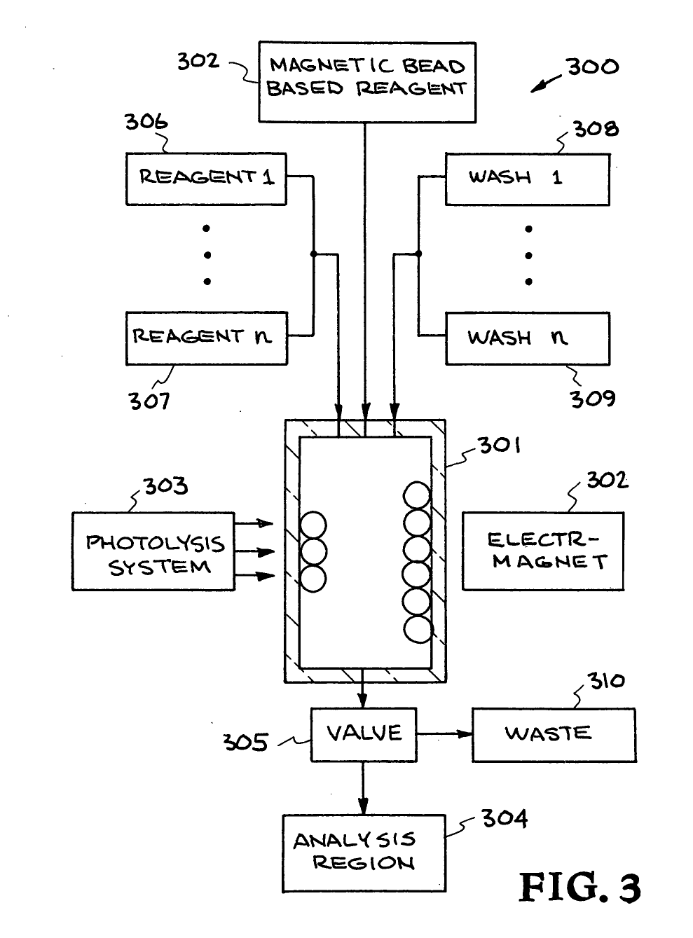

Referring now to FIG. 3, another embodiment of an immunoassay device constructed in accordance with the present invention is illustrated. The immunoassay device is indicated generally by reference numeral 300. The immunoassay device 300 is a device that provides biochemical assays. The immunoassay device 300 can, for example, be the type of immunoassay device described and illustrated in U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005. U.S. Patent Application No. 2005/0239192 by Shanavaz L. Nasarabadi, Richard G. Langlois, Billy W. Colston, Evan W. Skowronski, and Fred P. Milanovich for a hybrid automated continuous nucleic acid and protein analyzer using real-time PCR and liquid bead arrays; published Oct. 27, 2005 is incorporated herein by this reference.

The immunoassay device 300 utilizes a channel 301 through which fluids can be transported. A magnet 302 is positioned adjacent the channel 301. The magnet 302 can be an electromagnet. The electromagnet may be composed of any magnetic core material surrounded by a coil that can conduct an electrical current. The magnet 302 can be activated using electro/mechanical actuation. The channel 301 and magnet 302 provide an immunoassay magnetic trapping device. A photolysis unit 303 is positioned adjacent the channel 301 proximate the magnet 302.

A magnetic bead based reagent delivery unit 302 directs a magnetic bead based reagent into the channel 301. A sample is also directed into the channel 301 by the sample delivery unit. An individual reagent, or a reagent mix, is also directed into the channel 301. The reagent, or reagent mix, is produced by reagent delivery unit 306 for delivering Reagent 1 and/or reagent delivery unit 307 for delivering reagent n. The units 306 and 307 allow an individual reagent or a reagent mix comprising reagents 1 through reagent n to be delivered into channel 301. It is to be understood that additional reagent delivery units for delivering additional reagents can be added.

Signal molecules are connected to the beads providing a bead signal molecule combination 311. The signal molecules can, for example, comprise eTags available from Monogram Biosciences, Inc., 345 Oyster Point Blvd., South San Francisco, Calif. 94080-1913. The signal molecules 311 can be other signal molecules custom made or commercially available. The signal molecules 311 are released from trapped reagents using a cleaving process based on one or more physical or chemical processes.

A valve 305 downstream of the trapping region directs the flow of reagents to a waste stream 310, to an analysis unit 304, or to some other process area. The analysis unit 304 is a device that provides a bio-analysis. Detection of the signal molecules 311 is performed using any type of physical or chemical process, including but not limited to fluorescence, absorption, light scattering, electrochemical processes, conductivity, or mass spectrometry. The analysis unit 304 can, for example, be the type of device described and illustrated in U.S. Pat. No. 6,905,885 by Billy W. Colston, and Jonathan N. Simon for a portable pathogen detection system issued Jun. 14, 2005. U.S. Pat. No. 6,905,885 by Billy W. Colston, Matthew Everett, Fred P. Milanovich, Steve B. Brown, Kodumudi Venkateswaran, and Jonathan N. Simon for a portable pathogen detection system issued Jun. 14, 2005 is incorporated herein by this reference.

A wash, or a wash mix, is also directed into the channel 301. The wash, or wash mix, is produced by wash unit 308 (Wash 1) and/or other wash unit and/or wash unit 309 (Wash n). The units 308 and 309 allow an individual wash or a wash mix comprising wash 1 through wash n to be delivered into channel 301.

The immunoassay device 300 utilizes the channel 301 through which the fluids are transported. The magnet 302 is positioned adjacent the channel 301. The channel 101 and magnet 302 provide an immunoassay magnetic trapping device. The immunoassay device 300 allows biological assays to be performed using a bead based format. In the past, these were most frequently done in a static, batch configuration and exchange of reagents and washing steps performed manually. Each of the steps can dilute a sample so that the limit of detection for an assay is adversely affected. In the immunoassay device 300 flow through the magnetic trap allows rapid, efficient capture of magnetic bead based reagents, and can be used for pre concentration and sample clean up. Reagents and wash fluids flow past the captured sample and are sent to waste so that no dilution occurs in the assay. After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be removed from the trapping region by withdrawing the permanent magnet or shutting off the electromagnet. Spent magnetic beads can then be flushed from the trapping region using a pressure driven or electrophoretic flow. Removal of the beads prepares the system for another analysis with little cross contamination between samples.

The structural details of the immunoassay magnetic trapping device 100 having been described, the operation of the immunoassay magnetic trapping device 300 will now be considered. Flow through the immunoassay magnetic trapping device 300 allows rapid, efficient capture of magnetic bead based reagents 302, and can be used for pre concentration and sample clean up. Reagents 306 through 307 and wash fluids 308 through 309 can flow past the captured sample, beads/signal molecules 311 and be sent to waste 302 so that no dilution occurs in the assay. It is to be understood that between 306 and 307 or 308 and 309 any number of additional fluid steps can be included.

After performing a number of reaction and washing steps, eTags or other signal molecules that had been immobilized on the trapped beads can be released using a chemical or photolytic cleavage and directed to an analysis region. Signal molecules allow detection of species that themselves may not be easily detectible or are contained in an impure sample. The magnetic field can be “removed” or “withdrawn” as needed. Spent magnetic beads can then be flushed from the trapping region using a pressure driven or electrophoretic flow. Removal of the beads prepares the system for another analysis with little cross contamination between samples.

The general processes of the immunoassay magnetic trapping device 300 are the following:
1). Reagents immobilized on magnetic beads flow into the magnetic trap region. In the case of an immunoassay, the immobilized reagent is an antibody. With the magnetic field turned on in the trapping region, beads are removed from solution and captured.
2). A sample stream flows past the captured, immobilized reagents. Molecules with an affinity for the immobilized reagents, antigens in the immunoassay case, will be captured. Those that do not have such affinity will flow to waste. A large volume of sample can be processed in this way with the molecules of interest being captured and concentrated in a small volume.
3). Additional reactive streams are introduced into the trapping region. In the case of an eTag based immunoassay, this could be an eTag bound to an antibody. Alternatively, the immobilized reagents can be washed with water or other fluids to improve the stringency of the assay.
4). Any number of reactive streams or wash steps similar to 3) can be carried out.
5). Signal molecules are removed from the trapped reagents by a cleaving step and sent to the analysis region. For example, eTags can be freed by exposing the immobilized reagent complex to 680 nm light and sent to a capillary electrophoresis, laser induced fluorescence detection system.
6). The magnetic field is removed from the trapping region and all reagents are flushed to waste. The magnetic field is removed by translating the permanent magnet away from the flow channel or turning off the current in the electromagnet. Channels can be rinsed with water, bleach, detergent, or other cleaning fluids to minimize sample cross contamination. The system can then perform another analysis.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. An immunoassay apparatus for assaying a sample, comprising:
a channel,
magnetic beads,
signal molecules connected to said beads,
a magnetic bead based reagent delivery unit connected to said channel that delivers said magnetic beads and said signal molecules to said channel,
a magnet operatively connected to said channel,
a lysis unit connected to said channel,
a sample delivery unit connected to said channel,
at least one reagent delivery unit connected to said channel,
at least one wash delivery unit connected to said channel, and
an analysis unit connected to said channel.

2. The immunoassay apparatus of claim 1 wherein said magnet is a retractable magnet.

3. The immunoassay apparatus of claim 1 wherein said magnet is an electomagnet.

4. The immunoassay apparatus of claim 1 wherein said lysis unit is a photolysis unit.

5. The immunoassay apparatus of claim 1 wherein said lysis unit is a reagent lysis unit.

6. The immunoassay apparatus of claim 1 wherein said signal molecules are eTags.

7. A method of immunoassaying a sample, comprising the steps of:
providing magnetic beads,
connecting signal molecules to said beads,
connecting said sample to said magnetic beads with said connected signal molecules,
magnetically trapping said magnetic beads with said connected signal molecules and said sample,
lysising said sample, and
analyzing said sample.

8. The method of immunoassaying a sample of claim 7 wherein said step of magnetically trapping said magnetic beads comprises positioning a retractable magnet proximate said magnetic beads with said connected signal molecules and said sample.

9. The method of immunoassaying a sample of claim 7 wherein said step of magnetically trapping said magnetic beads comprises positioning an electomagnet proximate said magnetic beads with said connected signal molecules and said sample.

10. The method of immunoassaying a sample of claim 7 wherein said step of lysising said sample comprises photolysising said sample.

11. The method of immunoassaying a sample of claim 7 wherein said step of lysising said sample comprises reagent lysising said sample.

12. The method of immunoassaying a sample of claim 7 wherein said step of connecting signal molecules to said beads comprises connecting eTags to said beads.
